(19) United States
(12) Patent Application Publication
(10) Pub. No.: US 2026/0209182 A1
(43) Pub. Date: Jul. 23, 2026

(54) CRYSTALLINE FORMS OF N-(4-(6,7-DIMETHOXYQUINOLIN-4-YLOXY)PHENYL)-N'-(4-FLUOROPHENYL) CYCLOPROPANE-1,1-DICARBOXAMIDE FUMARATE AND PROCESS FOR ITS PREPARATION

(71) Applicant: MSN LABORATORIES PRIVATE LIMITED, R&D CENTER, Hyderabad, Telangana (IN)

(72) Inventors: Thirumalai Rajan SRINIVASAN, Hyderabad, Telangana (IN); Eswaraiah SAJJA, Hyderabad, Telangana (IN); Vijayavitthal t MATHAD, Hyderabad, Telangana (IN); Venkata Narasayya SALADI, Hyderabad, Telangana (IN); Bal Raju KAMMARI, Hyderabad, Telangana (IN); Srinivas Reddy GADE, Hyderabad, Telangana (IN); Venkateshwarlu SURIGILLA, Hyderabad, Telangana (IN); Srikanth PAREESHA, Hyderabad, Telangana (IN)

(73) Assignee: MSN LABORATORIES PRIVATE LIMITED, R&D CENTER, Hyderabad, Telangana (IN)

(21) Appl. No.: 19/132,820

(22) PCT Filed: Nov. 27, 2023
(86) PCT No.: PCT/IN2023/051104

§ 371 Date: May 26, 2025

(30) Foreign Application Priority Data
Nov. 25, 2022 (IN) 202241068017

(51) Int. Cl. C07D 215/233; A61K 31/47; C07C 57/15

(52) U.S. Cl. CPC C07D 215/233; A61K 31/47; C07C 57/15; C07B 2200/13

(57) ABSTRACT

The present invention relates to crystalline forms of N-(4-(6,7-dimethoxyquinolin-4-yloxy)phenyl)-N′-(4-fluorophenyl)cyclopropane-1,1-dicarboxamide fumarate represented by the following structural formula-1, and process for its preparation which is referred to as Cabozantinib fumarate.

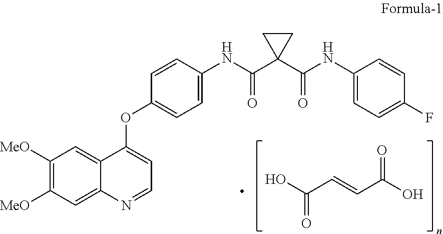

wherein n=0.25 to 2.0

### RELATED APPLICATION

This application claims the benefit of priority of our Indian patent application number 202241068017 filed on 25 Nov. 2022, which is incorporated herein by reference.

### FIELD OF THE INVENTION

The present invention relates to crystalline forms of N-(4-(6,7-dimethoxyquinolin-4-yloxy)phenyl)-N′-(4-fluorophenyl)cyclopropane-1,1-dicarboxamide fumarate represented by the following structural formula-1, which is referred to as Cabozantinib fumarate.

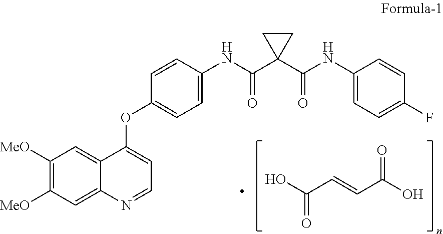

wherein n=0.25 to 2.0

### BACKGROUND OF THE INVENTION

Cabozantinib (S)-malate is a kinase inhibitor indicated for the treatment of patients with progressive, metastatic medullary thyroid cancer and advanced renal cell carcinoma in people who have received prior anti-angiogenic therapy.

Cabozantinib has been approved by the USFDA as COMETRIQ and CABOMETYX for the treatment of patients with progressive, metastatic medullary thyroid cancer and for the treatment of patients with advanced renal cell carcinoma (RCC) who have received prior antiangiogenic therapy with progressive, metastatic medullary thyroid cancer respectively.

COMETRIQ has also been approved by the European Commission for the treatment of adult patients with progressive, unresectable locally advanced or metastatic medullary thyroid carcinoma.

International (PCT) publication No. WO2005030140 A1 discloses Cabozantinib or a pharmaceutically acceptable salt and process for its preparation.

International (PCT) publication No. WO2010083414 A1 (hereinafter referred as WO '414) discloses amorphous forms of Cabozantinib L- and D-malate as well as the N-1 and N-2 polymorphs of crystalline Cabozantinib L- and D-malate and process for its preparation.

WO′414 also discloses water solubility of the various salts of Cabozantinib.

Polymorphism is the occurrence of different crystalline forms of a single compound and it is a property of some compounds and complexes. Thus, polymorphs are distinct solids sharing the same molecular formula, yet each polymorph may have distinct physical properties. Therefore, a single compound may give rise to a variety of polymorphic forms where each form has different and distinct physical properties, such as different solubility profiles, different melting point temperatures and/or different X-ray diffraction peaks. Since the solubility of each polymorph may vary, identifying the existence of pharmaceutical polymorphs is essential for providing pharmaceuticals with predicable solubility profiles. It is desirable to investigate all solid-state forms of a drug, including all polymorphic forms, and to determine the stability, dissolution and flow properties of each polymorphic form.

Polymorphic forms of a compound can be distinguished in a laboratory by X-ray diffraction spectroscopy and by other methods such as, infrared spectrometry. Additionally, polymorphic forms of the same drug substance or active pharmaceutical ingredient, can be administered by itself or formulated as a drug product (also known as the final or finished dosage form), and are well known in the pharmaceutical art to affect, for example, the solubility, stability, flowability, tractability and compressibility of drug substances.

### BRIEF DESCRIPTION OF THE INVENTION

The present invention relates to crystalline forms of Cabozantinib fumarate.

The present invention also relates to a process for the preparation of Cabozantinib fumarate.

### BRIEF DESCRIPTION OF DRAWINGS

FIG. 1: Illustrates the PXRD pattern of crystalline Form-S of Cabozantinib fumarate.

FIG. 2: Illustrates the PXRD pattern of crystalline Form-N of Cabozantinib fumarate.

FIG. 3: Illustrates the PXRD pattern of crystalline Form-M1 of Cabozantinib fumarate.

FIG. 4: Illustrates the PXRD pattern of crystalline Form-M2 of Cabozantinib fumarate.

FIG. 5: Illustrates the PXRD pattern of crystalline Form-M3 of Cabozantinib fumarate.

FIG. 6: Illustrates the PXRD pattern of crystalline Form-M4 of Cabozantinib fumarate.

FIG. 7: Illustrates the PXRD pattern of crystalline Form-M5 of Cabozantinib fumarate.

FIG. 8: Illustrates the PXRD pattern of crystalline Form-M6 of Cabozantinib fumarate.

FIG. 9: Illustrates the PXRD pattern of crystalline Form-N1 of Cabozantinib fumarate.

### DETAILED DESCRIPTION OF THE INVENTION

As used herein the term “suitable solvent” used in the present invention refers to “hydrocarbon solvents” such as n-hexane, n-heptane, cyclohexane, pet ether, toluene, pentane, cycloheptane, methyl cyclohexane, m-, o-, or p-xylene, and the like; “ether solvents” such as dimethoxy methane, tetrahydrofuran, 1,3-dioxane, 1,4-dioxane, diethyl ether, ethylene glycol dimethyl ether, ethylene glycol diethyl ether, diethylene glycol dimethyl ether, diethylene glycol diethyl ether, triethylene glycol dimethyl ether, anisole, t-butyl methyl ether, dimethoxy ethane and the like; “ester solvents” such as methyl acetate, ethyl acetate, isopropyl acetate, n-butyl acetate and the like; “polar-aprotic solvents such as dimethylacetamide (DMA), dimethylformamide (DMF), dimethylsulfoxide (DMSO), N-methylpyrrolidone (NMP) and the like; “chloro solvents” such as dichloromethane, dichloroethane, chloroform, carbon tetrachloride and the like; “ketone solvents” such as acetone, methyl ethyl ketone, methyl isobutylketone and the like; “nitrile solvents” such as acetonitrile, propionitrile, isobutyro nitrile and the like; “alcoholic solvents” such as methanol, ethanol, n-propanol, isopropanol, n-butanol, isobutanol, t-butanol, 2-nitroethanol, 2-fluoroethanol, 2,2,2-trifluoroethanol, ethylene glycol, 2-methoxyethanol, 1,2-ethoxyethanol, diethylene glycol, 1, 2, or 3-pentanol, neo-pentyl alcohol, t-pentyl alcohol, diethylene glycol, monoethyl ether, cyclohexanol, benzyl alcohol or glycerol and the like; “polar solvents” such as water or mixtures thereof.

The “suitable base” as used in the present invention is selected from inorganic bases like “alkali metal hydroxides” such as lithium hydroxide, sodium hydroxide, potassium hydroxide and the like; “alkali metal carbonates” such as sodium carbonate, potassium carbonate, lithium carbonate and the like; “alkali metal bicarbonates” such as sodium bicarbonate, potassium bicarbonate, lithium bicarbonate and the like; “alkali metal hydrides” such as sodium hydride, potassium hydride, lithium hydride and the like; ammonia; and organic bases such as “alkali metal alkoxides” such as sodium methoxide, sodium ethoxide, sodium tert-butoxide, potassium methoxide, potassium ethoxide, potassium tert-butoxide and the like; triethyl amine, methyl amine, ethylamine, 1,8-diazabicyclo[5.4.0]undec-7-ene (DBU), 1,5-diazabicyclo(4.3.0)non-5-ene (DBN), lithiumdiisopropylamide (LDA), n-butyl lithium, tribenzylamine, isopropyl amine, diisopropylamine, diisopropylethylamine (DIPEA), N-methylmorpholine (NMO), 4-methoxypyridine, N-ethylmorpholine, piperidine, dimethyl aminopyridine, morpholine, pyridine, 2,6-lutidine, 2,4,6-collidine, imidazole, 1-methyl imidazole, 1,2,4-triazole, 1,4-diazabicyclo[2.2.2]octane (DABCO) or mixtures thereof.

In the first embodiment, the present invention provides a crystalline Form-S of Cabozantinib fumarate of formula-1.

The first aspect of the first embodiment, the present invention provides a crystalline Form-S of Cabozantinib fumarate is characterized by its X-ray powder diffraction (PXRD) pattern as illustrated in FIG. 1.

In the second embodiment, the present invention provides a process for the preparation of crystalline Form-S of Cabozantinib fumarate of formula-1, which comprises:
a) contacting Cabozantinib to a solution of fumaric acid in water; and
b) isolating crystalline Form-S of Cabozantinib fumarate of formula-1.

In the process of second embodiment, adding Cabozantinib to a solution of fumaric acid at a suitable temperature such as about 25° C. to about the reflux temperature of the solvent used.

In the third embodiment, the present invention provides a crystalline Form-N of Cabozantinib fumarate of formula-1, is characterized by its X-ray powder diffraction (PXRD) pattern having 2 theta values at about 21.1 and 26.5±0.2 degrees.

In the first aspect of the third embodiment, the present invention provides a crystalline Form-N of Cabozantinib fumarate of formula-1, is further characterized by its X-ray powder diffraction (XRD) pattern having 2 theta values at about 6.3, 9.3, 9.6, 11.9, 12.8, 13.9, 14.8, 15.6, 18.1, 18.8 and 23.9±0.2 degrees.

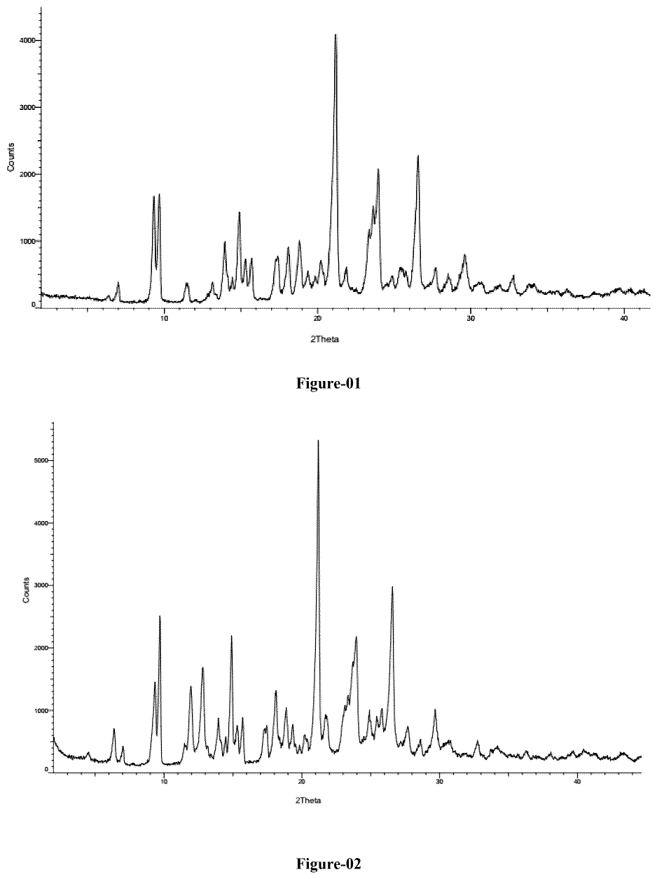

In the second aspect of the third embodiment, the present invention provides a crystalline Form-N of Cabozantinib fumarate of formula-1, is further characterized by the X-ray powder diffraction (PXRD) pattern as illustrated in FIG. 2.

In the fourth embodiment, the present invention provides a process for the preparation of crystalline Form-N of Cabozantinib fumarate of formula-1, which comprises:
a) contacting Cabozantinib fumarate with water; and
b) isolating crystalline Form-N of Cabozantinib fumarate of formula-1.

In the fifth embodiment, the present invention provides a process for the preparation of crystalline Form-N of Cabozantinib fumarate of formula-1, which comprises:
a) contacting Cabozantinib fumarate with tetrahydrofuran and ethyl acetate,
b) adding a suitable base to the mixture obtained in step-a) or adding the mixture obtained in step-a) to a suitable base,
c) adding a solution of fumaric acid in tetrahydrofuran and water to the mixture obtained in step-b) or adding the mixture obtained in step-b) to a solution of fumaric acid in tetrahydrofuran and water,
d) distilling off the solvent completely from the mixture obtained in step-c),
e) contacting water to the compound obtained in step-d), and
f) isolating crystalline Form-N of Cabozantinib fumarate of formula-1.

In the process of the fifth embodiment, the suitable base used in step-a) is selected from inorganic base or organic base.

In the process of the fifth embodiment, optionally the mixture obtained in step-b) can be filtered to make it particle free.

In the process of the fifth embodiment, adding water to the compound obtained in step-d) and optionally heating the mixture to 40-80° C.

In the first aspect of the fifth embodiment, a process for the preparation of Crystalline Cabozantinib fumarate, which comprises:
a) obtaining a mixture of Cabozantinib fumarate in a solvent; and
b) isolating crystalline Cabozantinib fumarate having PXRD pattern as illustrated in FIG. 2.

In the second aspect of the fifth embodiment, a process for the preparation of Crystalline Cabozantinib fumarate, which comprises:
a) obtaining a mixture of cabozantinib fumarate in a solvent comprising tetrahydrofuran, ethyl acetate and water,
b) distilling off solvent completely from the mixture obtained in step-a),
c) stirring the compound obtained in step-b) in water, and
d) isolating crystalline Cabozantinib fumarate having PXRD pattern as illustrated in FIG. 2.

The crystalline form-N of Cabozantinib fumarate obtained according to the present invention is having a purity of about 97% by HPLC or purity of about 99.0% by HPLC or purity of about 99.5% or purity of about 99.9% by HPLC.

In the sixth embodiment, the present invention provides a process for the preparation of a crystalline form of Cabozantinib fumarate, which comprises forming a solution of Cabozantinib fumarate and crystallizing the said Cabozantinib fumarate from the solution by precipitation, slurry or recrystallization.

In the process of the sixth embodiment, the wherein forming the solution of Cabozantinib fumarate comprises by suspending or dissolving the Cabozantinib fumarate in a suitable solvent.

The suitable solvent is selected from alcohol solvents, ester solvents, hydrocarbon solvents, nitrile solvents, polar-aprotic solvents, ketone solvents, ether solvents, chloro solvents, and water or mixture thereof.

In the seventh embodiment, the present invention provides a process for the preparation of Cabozantinib fumarate of formula-1, which comprises reacting compound of formula-2 with compound of formula-3 in the presence of a suitable base in a suitable solvent and then treating the obtained compound with fumaric acid in a suitable solvent to provide Cabozantinib fumarate of formula-1.

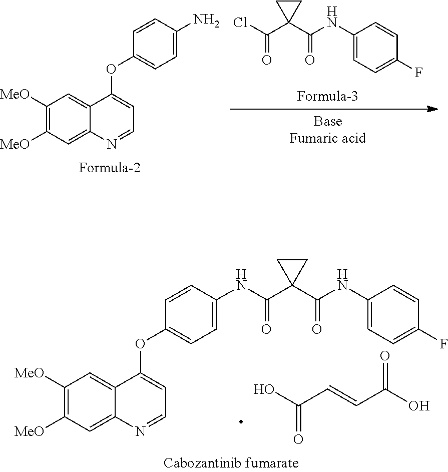

In the process of the seventh embodiment, the suitable base is selected from inorganic base or organic base; the suitable solvent is selected from alcohol solvents, ester solvents, hydrocarbon solvents, nitrile solvents, polar-aprotic solvents, ketone solvents, ether solvents, chloro solvents, and water or mixture thereof.

In the eighth embodiment, the present invention provides a crystalline Form-M1 of Cabozantinib fumarate.

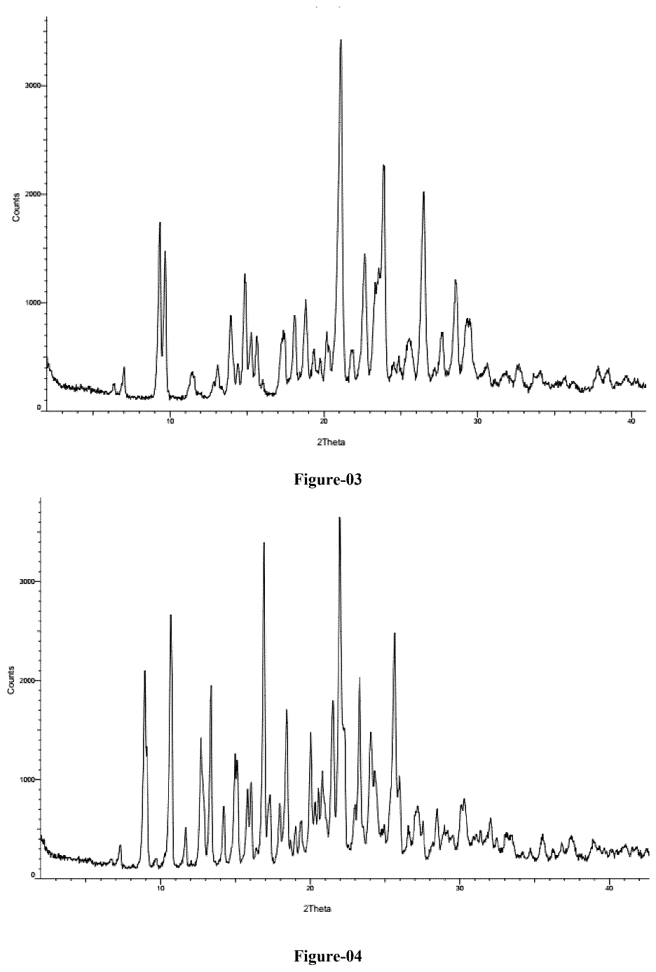

The crystalline Form-M1 of Cabozantinib fumarate is characterized by the X-ray powder diffraction (PXRD) pattern as illustrated in FIG. 3.

In the ninth embodiment, the present invention provides a process for the preparation of crystalline form-M1 of Cabozantinib fumarate, which comprises:
a) reacting compound of formula-2 with compound of formula-3 in the presence of a suitable base in a suitable solvent, and
b) treating the compound obtained in step-a) with fumaric acid,
c) isolating to provide crystalline Form-M1 of Cabozantinib fumarate of formula-1.

In the process of the ninth embodiment, treating the obtained compound with fumaric acid at a suitable temperature ranging from 25° C. to the reflux temperature of the solvent used to provide crystalline Form-M1 of Cabozantinib fumarate of formula-1.

In the process of the ninth embodiment, the suitable base used in step-a) is selected from organic base or inorganic base and suitable solvent used is selected from ester solvents, ether solvents, water or mixture thereof.

In the tenth embodiment, the present invention provides a crystalline Form-M2 of Cabozantinib fumarate.

The crystalline Form-M2 of Cabozantinib fumarate is characterized by the X-ray powder diffraction (PXRD) pattern as illustrated in FIG. 4.

In the eleventh embodiment, the present invention provides a process for the preparation of crystalline Form-M2 of Cabozantinib fumarate, which comprises:
a) contacting Cabozantinib fumarate with tetrahydrofuran and water; and
b) isolating crystalline Form-M2 of Cabozantinib fumarate.

In the process of the eleventh embodiment, adding tetrahydrofuran to Cabozantinib fumarate and heating the mixture to a temperature ranging from 40° C.-60° C.

In the process of the eleventh embodiment, further adding water to the mixture at 40° C.-60° C. and isolating the crystalline Form-M2 of Cabozantinib fumarate from the mixture by cooling the mixture 25-30° C.

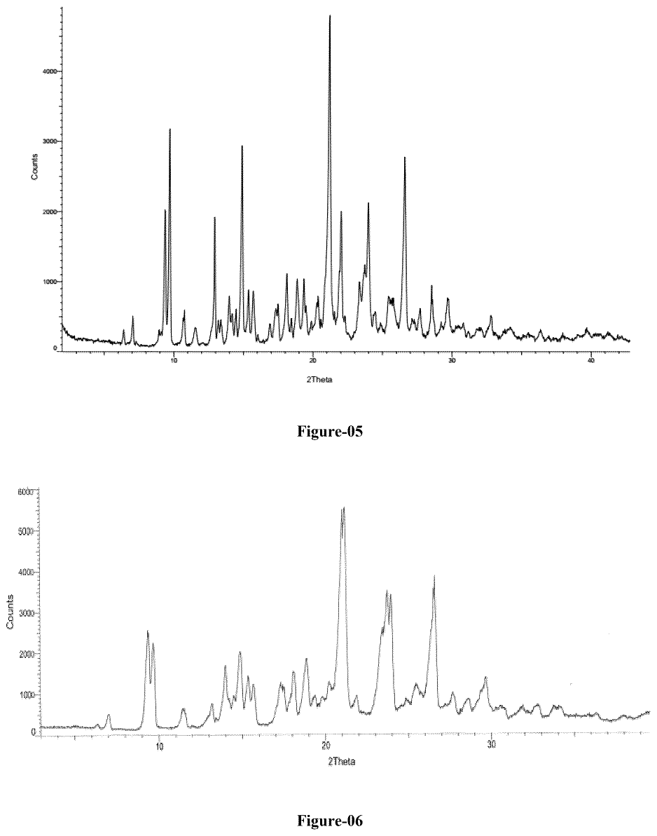

In the twelfth embodiment, the present invention provides a crystalline Form-M3 of Cabozantinib fumarate is characterized by its X-ray powder diffraction (PXRD) pattern as illustrated in FIG. 5.

In the thirteenth embodiment, the present invention provides a crystalline Form-M4 of Cabozantinib fumarate of formula-1.

The first aspect of the thirteenth embodiment, the present invention provides a crystalline Form-M4 of Cabozantinib fumarate is characterized by its X-ray powder diffraction (PXRD) pattern as illustrated in FIG. 6.

In the fourteenth embodiment, the present invention provides a process for the preparation of crystalline Form-M4 of Cabozantinib fumarate of formula-1, which comprises:
a) contacting Cabozantinib to a solution of fumaric acid in water; and
b) isolating crystalline Form-M4 of Cabozantinib fumarate of formula-1.

In the process of the fourteenth embodiment, preparing a solution of fumaric acid in water comprises adding water to fumaric acid and hearting the mixture to 75° C.-80° C., optionally filtering the mixture to make it particle free.

In the process of the fourteenth embodiment, adding the above mixture to Cabozantinib and heating to 75° C.-80° C. and isolating to provide crystalline Form-M4 of Cabozantinib fumarate.

In the fifteenth embodiment, the present invention provides a crystalline Form-M5 of Cabozantinib fumarate of formula-1.

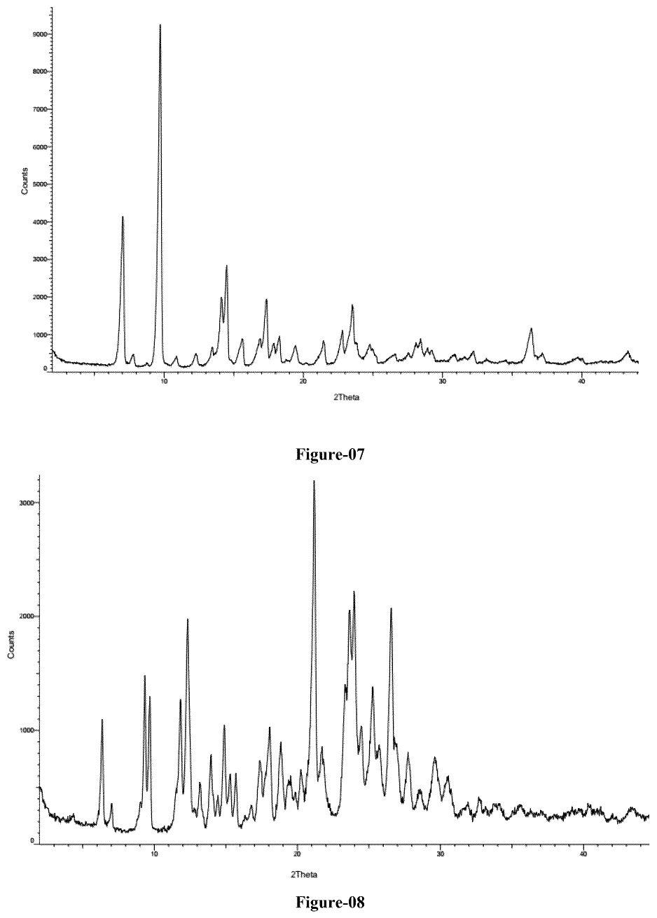

The first aspect of the fifteenth embodiment, the present invention provides a crystalline Form-M5 of Cabozantinib fumarate is characterized by its X-ray powder diffraction (PXRD) pattern as illustrated in FIG. 7.

In the sixteenth embodiment, the present invention provides a process for the preparation of crystalline Form-M5 of Cabozantinib fumarate of formula-1, which comprises:
a) contacting Cabozantinib fumarate with a suitable solvent; and
b) isolating crystalline Form-M5 of Cabozantinib fumarate of formula-1.

In the process of the sixteenth embodiment, the suitable solvent used in step-a) is selected from methanol, ethanol, propanol, isopropanol, butanol, isobutanol or mixture thereof.

In the process of the sixteenth embodiment, adding alcohol solvent to Cabozantinib fumarate and heating the mixture to 60-65° C. and isolating the crystalline Form-M5 of Cabozantinib fumarate by cooling the mixture to 25-30° C.

In the seventeenth embodiment, the present invention provides a crystalline Form-M6 of Cabozantinib fumarate of formula-1.

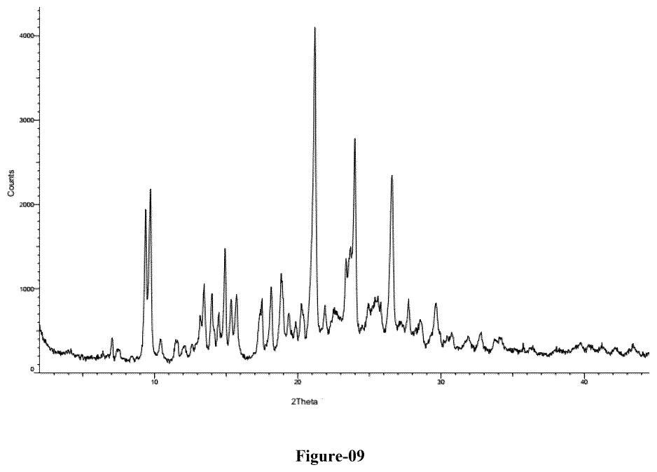

The first aspect of the seventeenth embodiment, the present invention provides a crystalline Form-M6 of Cabozantinib fumarate is characterized by its X-ray powder diffraction (PXRD) pattern as illustrated in FIG. 8.

In the eighteenth embodiment, the present invention provides a process for the preparation of crystalline Form-M6 of Cabozantinib fumarate of formula-1, which comprises:
a) contacting Cabozantinib fumarate with water; and
b) isolating crystalline Form-M6 of Cabozantinib fumarate of formula-1.

In the nineteenth embodiment, the present invention provides a process for the preparation of Cabozantinib fumarate of formula-1, which comprises reacting compound of formula-2 with compound of formula-4 in the presence of a suitable coupling agent in a suitable base and in a suitable solvent and then treating the obtained compound with fumaric acid in a suitable solvent to provide Cabozantinib fumarate of formula-1.

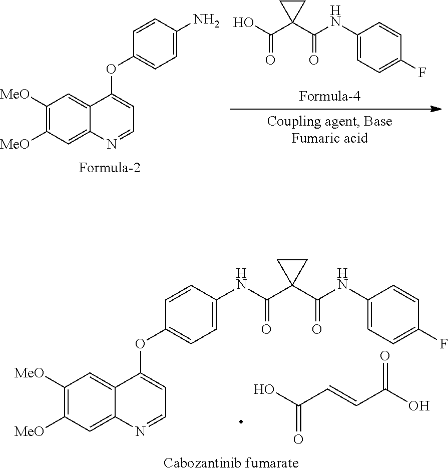

In the process of the nineteenth embodiment, wherein the suitable solvent is selected from alcohol solvents, ester solvents, hydrocarbon solvents, nitrile solvents, polar-aprotic solvents, ketone solvents, ether solvents, chloro solvents, and water or mixture thereof;
the suitable base is selected from inorganic base or organic base; the suitable coupling agent is selected from N,N′-dicyclohexylcarbodiimide (DCC), N,N′-diisopropylcarbodiimide (DIC), 1-ethyl-3-(3-dimethylaminopropyl) carbodiimide (EDCI), hydroxybenzotriazole (HOBt), 1-hydroxy-7-azabenzotriazole (HOAt), benzotriazole-1-yl-oxytripyrrolidino phosphoniumhexafluorophosphate (BOP reagent) benzotriazol-1-yl-oxytripyrrolidino phosphoniumhexafluorophosphate (PyBOP), 1-[bis(dimethylamino)methylene]-1H-1,2,3-triazolo[4,5-b]pyridinium 3-oxidhexafluorophosphate (HATU), O-(benzotriazol-1-yl)-N,N′,N′N′-tetramethyluroniumtetrafluoroborate (TBTU), N,N,N′,N-tetramethyl-O-(1H-benzo triazol-1-yl)uraniumhexafluorophosphate (HBTU), O-[(ethoxycarbonyl) cyanomethylen amino]-N,N′,N′,N′-tetramethyluroniumtetrafluoroborate (TOTU), or (1-cyano-2-ethoxy-2-oxoethylidenaminooxy)dimethylamino-morpholino-carbeniumhexafluorophosphate (COMU).

In an aspect, the present invention provides a process for the preparation of compound of formula-3, which comprises reacting compound of formula-4 with a suitable acylating agent in a suitable solvent to provide compound of formula-3.

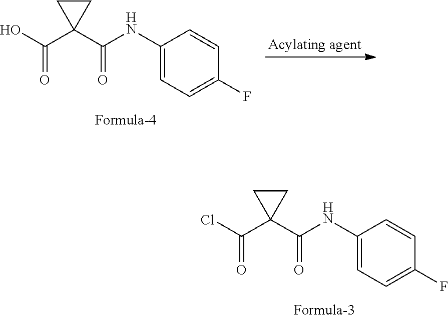 [SMILES: O=C(O)C1(C(=O)Nc2ccc(F)cc2)CC1 ; O=C(Cl)C1(C(=O)Nc2ccc(F)cc2)CC1]

The suitable acylating agent is selected from thionyl chloride, oxalyl chloride, phosphorus oxychloride, phosphorus trichloride, phosphorus pentachloride, sulfuryl chloride and phosgene and the suitable solvent is selected from alcohol solvents, ester solvents, hydrocarbon solvents, nitrile solvents, polar-aprotic solvents, ketone solvents, ether solvents, chloro solvents, and water or mixture thereof.

In the process of the present invention, isolating the crystalline forms of Cabozantinib fumarate can be carried out by any methods known in the art or crystalline forms of Cabozantinib fumarate can be isolated by employing any of the techniques, but not limited to: decantation, filtration by gravity or suction, centrifugation, adding solvent to make slurry followed by filtration, or other techniques specific to the equipment used and the like, and optionally washing with a solvent.

In the process of the present invention, the crystalline forms of Cabozantinib fumarate can be dried in a suitable drying equipment such as tray dryer, vacuum oven, rotatory cone dryer, air oven, fluidized bed dryer, spin flash dryer, flash dryer, or the like. The drying can be carried out at atmospheric pressure or under reduced pressure at temperature of less than about 100° C., less than about 60° C., less than about 40° C., or any other suitable temperature. The drying can be carried out for any time period required for obtaining a desired quality, such as from about 15 minutes to 10 hours or longer.

The compound of formulae 2, 4 and Cabozantinib used in the present invention can be synthesized from any of the prior known processes.

In the twentieth embodiment, Cabozantinib fumarate obtained according to the present invention has a particle size distribution of D90 less than 150 μm, preferably less than 100 μm; more preferably less than 50 μm.

Cabozantinib fumarate of formula-1 obtained according to the present invention contains fumaric acid, which is equivalent to 0.4 to 2.0 moles per mole of Cabozantinib free base.

The crystalline forms of Cabozantinib fumarate obtained according to the present invention is having a purity of about 97% by HPLC or purity of about 99.0% by HPLC or purity of about 99.5% or purity of about 99.9% by HPLC.

The crystalline forms of Cabozantinib fumarate obtained according to the present invention which are anhydrous or a hydrate thereof.

Crystalline forms of Cabozantinib fumarate prepared according to the present invention can be further micronized or milled in conventional techniques to get the desired particle size to achieve desired solubility profile based on different forms of pharmaceutical composition requirements. Techniques that can be used for particle size reduction include, but not limited to ball milling, roll milling and hammer milling, and jet milling. Milling or micronization can be performed before drying, or after the completion of drying of the product.

In another embodiment, the present invention provides pharmaceutical composition comprising crystalline forms of Cabozantinib fumarate and one or more pharmaceutically acceptable excipients.
wherein, the excipient can be selected from those reported in Excipient Development for Pharmaceutical, Biotechnology, and Drug Delivery Systems 2006.

In yet another embodiment, pharmaceutical composition comprising crystalline forms of Cabozantinib fumarate and one or more pharmaceutically acceptable excipients is formulated in a manner suitable for the route of administration to be used.

As used herein, the term “pharmaceutical compositions” include tablets, pills, powders, liquids, suspensions, emulsions, granules, capsules, suppositories, or injection preparations.

### P-XRD Method of Analysis:

PXRD analysis of crystalline forms of the present invention was carried out by using BRUKER/D8 ADVANCE diffractometer using Cu Kα radiation of wavelength 1.5406 A° and continuous scan speed of 0.03°/min.

### PSD Method of Analysis:

Particle size distribution (PSD) analysis was performed using Malvern Mastersizer 3000 instrument.

The process described in the present invention was demonstrated in examples illustrated below. These examples are provided as illustration only and therefore should not be construed as limitation of the scope of the invention.

### EXAMPLES

### Example-1: Preparation of Crystalline Form-S of Cabozantinib Fumarate

Water (1000.0 ml) was added to Fumaric acid (11.5 gms) at 25-30° C. and stirred for 50 minutes. Cabozantinib (25.0 gms) was added to the mixture at 25-30° C. and stirred for 4 hours. Filtered the mixture and dried to get the title compound. Yield: 33.2 gms.

The PXRD pattern of the obtained compound is shown in figure-1.

### Example-2: Preparation of Crystalline Form-N of Cabozantinib Fumarate

Water (320.0 ml) was added to Cabozantinib fumarate (8.0 gms) at 25-30° C. and stirred for 5 minutes. Heated the mixture to 45-50° C. and stirred for 4 hours. Filtered the solid and dried to get the title compound. Yield: 7.5 gms.

The PXRD pattern of the obtained compound is shown in figure-2.

### Example-3: Preparation of Crystalline Form-M1 of Cabozantinib Fumarate

Tetrahydrofuran (600.0 ml) was added to compound of formula-4 (113.0 gms) at 25-30° C. Thionyl chloride (180.7 gms) was slowly added to the mixture at 25-30° C. and stirred for 6 hours. The obtained mixture was slowly added to a pre-cooled mixture of tetrahydrofuran (600 ml), compound of formula-2 (100.0 gms), potassium carbonate (419.2 gms) and water (600 ml) at 0-5° C. Raised the temperature of the mixture to 25-30° C. and stirred for 2 hours. Layers were separated and aqueous layer extracted with ethyl acetate and water. Combined the total organic layers and washed with aqueous sodium bicarbonate solution. Layers were separated and organic layer washed with aqueous sodium chloride solution. Carbon was added to organic layer and stirred for 15 minutes. Filtered the organic layer through hyflow bed and washed with tetrahydrofuran. Fumaric acid (39.17 gms) was added to the filtrate at 25-30° C. Distilled off the solvent completely from the mixture at below 40° C. under vacuum to get the title compound.

The PXRD pattern of the obtained compound is illustrated in FIG. 3.

### Example-4: Preparation of Crystalline Form-M2 of Cabozantinib Fumarate

Tetrahydrofuran (500 ml) was added to the compound obtained in example-3 at 25-30° C. Raised the temperature of the mixture to 50-55° C. and stirred for 15 minutes. Water (1000 ml) was added to the mixture at 50-55° C. and stirred for 8 hours. Cooled the mixture to 25-30° C. and stirred for 2 hours. Filtered the solid and washed with tetrahydrofuran. Repeated the above process twice to get the title compound. Yield: 62.0 gms.

Fumaric acid content: 11.16%.
The PXRD pattern of the obtained compound is illustrated in FIG. 4.

### Example-5: Preparation of Crystalline Form-M3 of Cabozantinib Fumarate

Tetrahydrofuran (600 ml) was added to compound of formula-4 (113 gms) at 25-30° C. Thionyl chloride (180.7 gms) was slowly added to the mixture at 25-30° C. and stirred for 6 hours. The obtained mixture was slowly added to a pre-cooled mixture of tetrahydrofuran (600 ml), compound of formula-2 (100 gms), potassium carbonate (419.2 gms) and water (600 ml) at 0-5° C. Raised the temperature of the mixture to 25-30° C. and stirred for 2 hours. Layers were separated and aqueous layer extracted with water and ethyl acetate. Layers were separated and aqueous layer extracted with ethyl acetate. Layers were separated. Combined the total organic layers. Water was added to the organic layer and washed with acetic acid. Layers were separated and organic layer washed with aqueous sodium bicarbonate solution. Layers were separated. Carbon was added to organic layer and stirred for 30 minutes. Filtered the organic layer through hyflow bed and washed the bed with tetrahydrofuran. Fumaric acid (78.3 gms) was added to the filtrate at 25-30° C. and stirred for 10 minutes. Distilled off the solvent completely from the mixture at below 40° C. under vacuum. Tetrahydrofuran (1000 ml) was added to the obtained compound at 25-30° C. Raised the temperature of the mixture to 50-55° C. and stirred for 20 minutes. Water (2000 ml) was added to the mixture at 50-55° C. and stirred for 8 hours. Cooled the mixture to 25-30° C. and stirred for 2 hours. Repeated the same process again and filtered the solid, washed with tetrahydrofuran and dried to get the title compound. Yield: 128 gms.

Fumaric acid content: 20.50%.
The PXRD pattern of the obtained compound is illustrated in FIG. 5.

### Example-6: Preparation of Crystalline Form-N1 of Cabozantinib Fumarate

Tetrahydrofuran (700 ml) and ethyl acetate (300 ml) were added to Cabozantinib fumarate (100 gms) at 25-30° C. and stirred for 10 minutes. Aqueous sodium carbonate solution were added to the mixture at 25-30° C. and stirred for 20 minutes. Layers were separated and aqueous layer extracted with tetrahydrofuran and ethyl acetate. Layers were separated. Combined the total organic layers and washed with aqueous sodium metabisulfite solution. Layers were separated and organic layer washed with aqueous sodium chloride solution. Layers were separated. Carbon was added to organic layer and stirred for 20 minutes. Filtered the organic layer through hyflow bed and washed with tetrahydrofuran. A mixture of tetrahydrofuran (300 ml), water (20 ml) and fumaric acid (22.55 gms) were added to the filtrate at 25-30° C. and stirred for 20 minutes. Distilled off the solvent completely from the mixture at below 40° C. under vacuum to get the title compound.

The PXRD pattern of the obtained compound is illustrated in FIG. 9.

### Example-7: Preparation of Crystalline Form-N of Cabozantinib Fumarate

Water (5000 ml) was added to the compound obtained in example-6 at 25-30° C. and stirred for 1 hour. Raised the temperature of the mixture to 50-55° C. and stirred for 12 hours. Cooled the mixture to 25-30° C. and stirred for 2 hours. Filtered the solid and washed with water. Water (2000 ml) was added to the obtained compound at 25-30° C. Raised the temperature of the mixture to 50-55° C. and stirred for 2 hours. Cooled the mixture to 25-30° C. and stirred for 2 hours. Filtered the solid, washed with water and dried to get the title compound. Yield: 88.0 gm. Fumaric acid content: 20.46%. Purity by HPLC: 99.92%.

Particle size distribution: D90: 81.47 μm. Water content: 2.15%.
The PXRD pattern of the obtained compound is illustrated in FIG. 2.

### Example-8: Preparation of Crystalline Form-M4 of Cabozantinib Fumarate

Water (500 ml) was added to fumaric acid (2.77 gms) at 25-30° C. Raised the temperature of the mixture to 80-85° C. and filtered the mixture for particle free. To the solution was added to the Cabozantinib at 25-30° C. and stirred for 10 minutes. Raised the temperature of the mixture to 80-85° C. and stirred for 19 hours. Filtered the mixture under vacuum and dried to get the title compound. Yield: 7.0 gms.

The PXRD pattern of the obtained compound is illustrated in FIG. 6.

### Example-9: Preparation of Crystalline Form-M5 of Cabozantinib Fumarate

Methanol (600 ml) was added to Cabozantinib fumarate (10 gms) at 25-30° C. Raised the temperature of the mixture to 60-65° C. and stirred for 1 hour. Cooled the mixture to 25-30° C. and stirred for overnight. Filtered the solid, washed with methanol and dried to get the title compound. Yield: 6.0 gms.

The PXRD pattern of the obtained compound is illustrated in FIG. 7.

### Example-10: Preparation of Crystalline Form-M6 of Cabozantinib Fumarate

Water (400 ml) was added to Cabozantinib fumarate (20 gms) at 25-30° C. and stirred for 1 hour at the same temperature. Filtered the solid, washed with water and dried to get the title compound. Yield: 17.0 gms.

The PXRD pattern of the obtained compound is illustrated in FIG. 8.

We claim:

1. Cabozantinib fumarate.

2. Crystalline form of Cabozantinib fumarate of formula-1, which is characterized by X-ray powder diffraction (PXRD) pattern having one or more 2-theta peaks at about 8.9, 9.6, 10.7, 11.9, 12.8, 13.4, 14.9, 15.7, 16.9, 18.1, 18.4, 21.1, 21.9, 23.9, 25.6, 26.5=0.2 degrees.

3. Crystalline form of Cabozantinib fumarate of claim 1 or 2, characterized by a PXRD pattern as illustrated in any of FIGS. 1-8 or mixtures thereof.

4. Crystalline form of Cabozantinib fumarate of claim 3, which is anhydrous or a hydrate thereof.

5. A process for the preparation of crystalline form of Cabozantinib fumarate of claim 2 or 3, which comprises obtaining a solution comprising Cabozantinib fumarate and isolating the crystalline form of Cabozantinib fumarate.

6. The process as claimed in claim 5 wherein obtaining a solution of Cabozantinib fumarate comprises suspending or dissolving Cabozantinib and fumaric acid, or Cabozantinib fumarate in a solvent.

7. The process as claimed in claim 6 wherein the solvent is selected from alcohol solvents, ester solvents, hydrocarbon solvents, nitrile solvents, polar-aprotic solvents, ketone solvents, ether solvents, chloro solvents, water and mixture thereof.

8. A process for the preparation of crystalline Cabozantinib fumarate, which comprises:
a) obtaining a mixture of Cabozantinib fumarate in a solvent; and
b) isolating crystalline Cabozantinib fumarate having PXRD pattern as illustrated in FIG. 2.

9. A process as claimed in claim 8, which comprises:
a) obtaining a mixture of Cabozantinib fumarate in a solvent comprising tetrahydrofuran, ethyl acetate and water,
b) distilling off solvent completely from the mixture obtained in step-a),
c) stirring the compound obtained in step-b) in water, and
d) isolating crystalline Cabozantinib fumarate having PXRD pattern as illustrated in FIG. 2.

10. The process as claimed in claim 9 wherein the step (c) comprises optional heating to 40-80° C.

11. A process for the preparation of Cabozantinib fumarate, which comprises:
a) reacting compound of formula-2 with compound of formula-3 in the presence of a base in a solvent,
b) treating the compound obtained in step-a) with fumaric acid, and
c) isolating Cabozantinib fumarate of formula-1.

12. The process as claimed in claim 11 wherein the Cabozantinib fumarate of formula 1 obtained in step (c) is crystalline Cabozantinib fumarate having PXRD pattern as illustrated in FIG. 3.

13. The process as claimed in claim 11 wherein,
the base used in step-a) is selected from inorganic base or organic base,
the solvent used in steps a) and b) is selected from ester solvent, ether solvent, water or mixture thereof.

14. The process as claimed in claim 11 wherein treating the compound obtained in step-a) with fumaric acid is at a suitable temperature ranging from 25° C. to the reflux temperature of the solvent used.

15. A process for the preparation of crystalline Cabozantinib fumarate, which comprises:
a) contacting Cabozantinib fumarate with a solvent, and
b) isolating crystalline Cabozantinib fumarate having PXRD pattern as illustrated in FIG. 4.

16. The process as claimed in claim 15 wherein the solvent used in step-a) is selected from tetrahydrofuran, water or mixture thereof,

17. The process as claimed in claim 15 wherein step-a) comprises heating the mixture to a temperature ranging from 40° C.-60° C.

18. A process for the preparation of crystalline Cabozantinib fumarate, which comprises:
a) contacting Cabozantinib fumarate with alcohol solvent; and
b) isolating crystalline Cabozantinib fumarate having PXRD pattern as illustrated in FIG. 7.

19. The process as claimed in claim 18 wherein the alcohol solvent is selected from methanol, ethanol, propanol, isopropanol, butanol, isobutanol or mixtures thereof.

20. The process as claimed in claim 18 wherein step (a) comprises adding alcohol solvent to Cabozantinib fumarate and heating the mixture to 60-65° C.

21. Cabozantinib fumarate having a particle size distribution of D90 less than 150 μm, preferably less than 100 μm, more preferably less than 50 μm.

22. Cabozantinib fumarate having a purity of about 99.5% by HPLC.

23. A pharmaceutical composition comprising Cabozantinib fumarate according to any of the preceding claims and one or more pharmaceutically acceptable excipients.

24. The use of Cabozantinib fumarate according to any of preceding claims for the preparation of a medicament for the treatment of patients with progressive, metastatic medullary thyroid cancer.

25. The use of Cabozantinib fumarate according to any of preceding claims for the preparation of a medicament for the treatment of patients with advanced renal cell carcinoma (RCC) who have received prior antiangiogenic therapy with progressive, metastatic medullary thyroid cancer.
